(19) United States
(12) Patent Application Publication
(10) Pub. No.: US 2019/0034556 A1
(43) Pub. Date: Jan. 31, 2019

(54) METHOD, APPARATUS AND REFRIGERATOR FOR RECIPE RECOMMENDATION

(71) Applicant: BOE TECHNOLOGY GROUP CO., LTD., Beijing (CN)

(72) Inventors: Yu GU, Beijing (CN); Hongli DING, Beijing (CN); Ying ZHANG, Beijing (CN); Kai ZHAO, Beijing (CN); Yifei ZHANG, Beijing (CN)

(21) Appl. No.: 15/956,687

(22) Filed: Apr. 18, 2018

(30) Foreign Application Priority Data
Jul. 31, 2017 (CN) 201710641577.4

(51) Int. Cl. G06F 17/30; G01G 19/414; G06K 9/62; G01N 33/02

(52) U.S. Cl. CPC G06F 17/30979; G01G 19/414; G06F 17/30867; G01N 33/02; G06K 9/6268

(57) ABSTRACT

The present disclosure proposes a recipe recommendation method, a recipe recommendation apparatus, and a refrigerator. The method includes acquiring a freshness of a candidate food material; classifying the candidate food material as a target food material or an inedible food material based on the freshness of the candidate food material; acquiring a candidate recipe corresponding to the target food material to generating a set of candidate recipes; calculating a score for the candidate recipes, the score indicating a degree to which the candidate recipe is recommended; determining a recommended recipe based on the score of the candidate recipe in the set of candidate recipes; and recommending the recommended recipe.

### RELATED APPLICATIONS

This application claims the priority of Chinese Patent Application No. 201710641577.4 filed on Jul. 31, 2017, the entire contents of which is incorporated herein by reference.

### TECHNICAL FIELD

This disclosure relates to the field of home appliance technology, and more particularly to a method, an apparatus, and a refrigerator for recipe recommendation.

### BACKGROUND

With the improvement of living standards, people's diet requirements are also getting higher and higher. It is desired to taste different kinds of dishes. Therefore, how to realize a personalized recommendation of recipes has gradually become a research hotspot.

However, the existing recipe recommendation method does not account for the food materials owned by users at hand and the freshness thereof, which may easily lead to a waste of food materials.

### SUMMARY

The present disclosure is intended to address at least one of the technical problems in the related technical field to some extent.

An embodiment of a first aspect of the present disclosure provides a recipe recommendation method, comprising:

acquiring a freshness of a candidate food material;

classifying the candidate food material as a target food material or an inedible food material based on the freshness of the candidate food material;

acquiring a candidate recipe corresponding to the target food material to generate a set of candidate recipes;

calculating a score for the candidate recipe, the score indicating a degree to which the candidate recipe is recommended;

determining a recommended recipe based on the score of the candidate recipe in the set of candidate recipes; and

recommending the recommended recipe.

It is to be understood that in any method claimed herein that includes more than one step or acts, the sequence of steps or acts of the method is not necessarily limited to the order in which the steps or acts of the method are recited, unless stated otherwise.

An embodiment of a second aspect of the present disclosure provides a recipe recommendation apparatus, comprising:

an acquisition module configured to acquire a freshness of a candidate food material;

a classification module configured to classify the candidate food material as a target food material or an inedible food material based on the freshness of the candidate food material;

a generation module configured to acquire a candidate recipe corresponding to the target food material to generate a set of candidate recipes;

a calculation module configured to calculate a score of the candidate recipes, the score indicating a degree to which the candidate recipe is recommended;

a determination module configured to determine a recommended recipe based on the score of the candidate recipe in the set of candidate recipes; and

a recommendation module for recommend the recommended recipe.

An embodiment of a third aspect of the present disclosure provides a refrigerator comprising at least one of a camera and an infrared sensor, a memory, a processor, and a computer program stored on the memory and executable on the processor, wherein:

the camera is configured to acquire a picture of a candidate food material;

the infrared sensor is configured to determine an infrared thermal energy on the candidate food material; and

the processor is configured to implement the recipe recommendation method as described in the embodiment of the first aspect of the disclosure by executing the computer program based on at least one of the picture acquired by the camera and the infrared thermal energy determined by the infrared sensor.

An embodiment of a fourth aspect of the present disclosure provides a non-transitory computer-readable storage medium storing thereon a computer program which, when executed by a processor, implements a recipe recommendation method as described in the embodiment of the first aspect of the present disclosure.

An embodiment of a fifth aspect of the present disclosure provides a computer program product that executes a recipe recommendation method as described in the embodiment of the first aspect embodiment of the present disclosure when an instruction in the computer program product is executed by a processor.

A part of the additional aspects and advantages of the disclosure will be set forth in the description below and, the other part will be apparent from the description below, or may be appreciated by practice of the disclosure.

### BRIEF DESCRIPTION OF THE DRAWINGS

The foregoing and additional aspects and advantages of the present disclosure will become apparent and readily understood from the following description of the embodiments, taken in conjunction with the accompanying drawings, in which:

FIG. 1 is a schematic flow chart of a recipe recommendation method according to an embodiment of the present disclosure;

FIG. 2 is a schematic flow chart of a method for acquiring a freshness of a candidate food material;

FIG. 3 is a schematic flow chart of another method for acquiring the freshness of the candidate food material;

FIG. 4 is a schematic flow chart of a recipe recommendation method according to another embodiment of the present disclosure;

FIG. 5 is a schematic histogram of user historical data established according to a taste dimension;

FIG. 6 is a schematic block diagram of a recipe recommendation apparatus according to an embodiment of the present disclosure;

FIG. 7 is a schematic block diagram of a recipe recommendation apparatus according to another embodiment of the present disclosure;

FIG. 8 is a schematic structural diagram of a recipe recommendation apparatus according to an embodiment of the present disclosure; and

FIG. 9 is a schematic structural diagram of a refrigerator according to an embodiment of the present disclosure.

### DETAILED DESCRIPTION

Embodiments of the present disclosure are described in detail below. Examples of the embodiments are shown in the drawings. In the drawings, the same or similar reference numbers indicate the same or similar elements or elements having the same or similar functions throughout. The embodiments described below with reference to the drawings are exemplary. These embodiments are intended to be used to explain the present disclosure and should not be construed as a limitation of the present disclosure.

The recipe recommendation method, the recipe recommendation device, and the refrigerator according to embodiments of the present disclosure are described below with reference to the accompanying drawings.

With the continuous improvement of the living standards, people's requirements for home appliances are also getting higher and higher. For example, users may expect the refrigerator to have a recipe recommendation function to recommend dishes that should be made to the users.

Currently, commercially available refrigerators with a recipe recommendation function normally are only able to provide a recipe browsing function, or only able to recommend recipes to users based on their preferences. Existing refrigerators have not yet taken into account the biological categories of food materials stored in the refrigerator and the freshness thereof. Therefore, some food materials may decompose for being preserved too long and thus being inedible. Eating these food materials may cause physical discomfort. Meanwhile, some food materials have been preserved for some period of time but have not decomposed. Therefore, these food materials are still edible if consumed immediately. Failing to consume these food materials timely may causes a waste.

In order to solve at least the above problems, an embodiment of the present disclosure provides a recipe recommendation method. The method according to the embodiment of the present disclosure facilitates a user to preferentially consume the food materials having a relatively low freshness but still edible by recommending to the user a recipe corresponding to such food materials, thus avoiding the waste of the food materials.

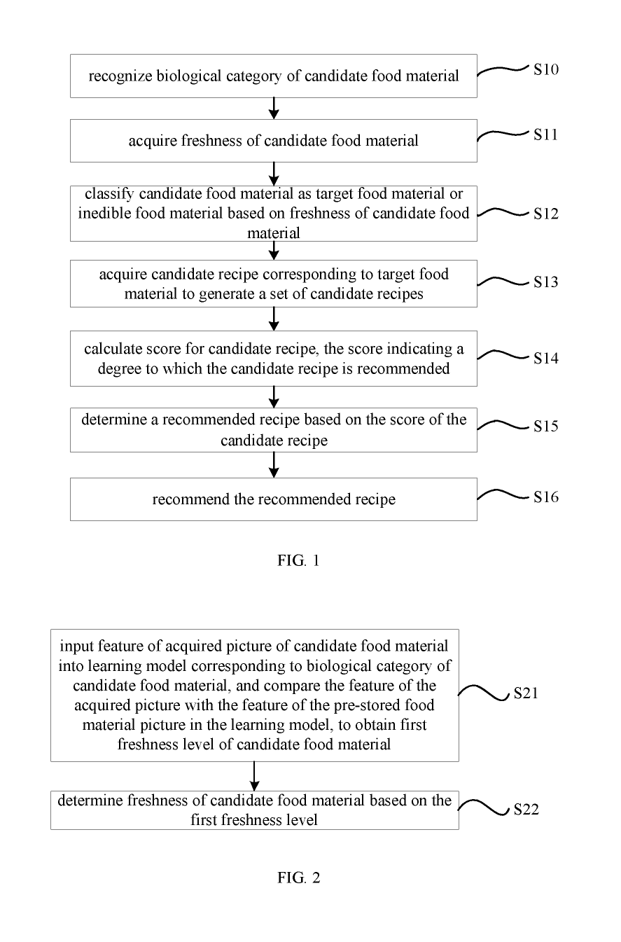

FIG. 1 is a schematic flow chart of a recipe recommendation method according to an embodiment of the present disclosure.

As shown in FIG. 1, the recipe recommendation method includes the following steps:

S11, acquiring a freshness of a candidate food material;

S12, classifying the candidate food material as a target food material or an inedible food material based on the freshness of the candidate food material;

S13, acquiring a candidate recipe corresponding to the target food material to generate a set of candidate recipes;

S14, calculating a score for each of the candidate recipes, the score indicating a degree to which the candidate recipe is recommended;

S15, determining a recommended recipe based on the score of the candidate recipe in the set of candidate recipes; and

S16, recommending the recommended recipe.

The recipe recommendation method above will be described in detail below.

In an embodiment of the present disclosure, the candidate food material is vegetable. It should be understood that vegetable is only an example of the candidate food material and that the candidate food material is not limited to vegetable. For example, the candidate food material can be fruit and meat. The present disclosure will be described by taking vegetables as examples.

A vegetable has higher nutrition when just picked. The nutrition in the vegetables drain away over time. The longer the preservation time after being picked, the more the nutrition lost. A longer preservation time can even cause decomposition of the vegetables.

Therefore, in order to prevent the user from eating food materials with low nutritional value and being discomfort due to consumption of the decomposed food materials, in an embodiment according to the present disclosure, the recipe recommendation method may include acquiring the freshness of one or more candidate food materials. To acquire the freshness of the candidate food materials, in some embodiments, the biological categories of the candidate food materials may be recognized firstly. For example, a vegetable may be classified into any of the following biological categories, which including but not limited to, root vegetables, Chinese cabbage vegetable, kale vegetables, solanaceous vegetable, leguminous plants, melon vegetables, aquatic plant and the like.

Refrigerator is a device for storing food materials. Putting the food materials into the refrigerator can reduce the loss speed of moisture and nutrition of the food materials. In this embodiment, a camera may be provided in the refrigerator. Firstly, a picture of the food materials stored in the refrigerator is collected by the camera, and then the biological category to which each candidate food material belongs is determined (step S10). As an example, pictures of food materials in the each biological category may be stored in a cloud server or a refrigerator memory in advance. The biological category of each candidate food material can be determined by uploading the picture of the candidate food material acquired by the camera to a processing unit of the refrigerator and comparing the picture of the candidate food material with the pre-stored pictures by the processing unit. The features used for the comparison of the pictures may include color, brightness, shape and the like of the objects in the pictures.

After determining the biological category of the candidate food material, the freshness of the candidate food material can be acquired (step S11).

In an embodiment, the freshness of the vegetable may be divided into a plurality of levels. For example, the freshness of the vegetable can be divided into four levels, namely level 1, level 2, level 3 and level 4. Vegetables with the freshness of level 1 are the freshest vegetables and can be stored for the longest time. Vegetables with the freshness of level 2 are relatively fresh and can still be stored for a certain period of time, despite the nutrition and water have been starting losing. Vegetables with the freshness of level 3 are less fresh. Although still edible, they have a limited storage time, and therefore should be recommended to be consumed immediately. Vegetables with the freshness of level 4 are the least fresh vegetables, and therefore are not recommended for consumption. In general, the freshness level is inversely proportional to the degree of freshness of the food materials. The higher the freshness level, the lower the degree of freshness of the food materials is. The lower the freshness level, the fresher the food materials are.

Other ways to divide the freshness levels are equally viable. For example, the freshness can be divided into 3 levels. Vegetables with the freshness of level 1 can be stored for a long term. Vegetables with the freshness of level 2 should be consumed as soon as possible. Vegetables with the freshness of level 3 are not edible. It should be understood that the above ways of dividing the freshness levels are merely exemplary.

In this embodiment, after acquiring the freshness of the candidate food material, the candidate food material can be classified as a target food material or an inedible food material according to the acquired freshness information. For example, in an embodiment, a food material with a higher freshness (i.e. with a lower freshness level) can be selected as the target food material. Specifically, when the freshness of a plurality of candidate food materials includes the above four levels of freshness, food materials with the freshness of levels 1-3 are selected as the target food materials since food materials with the freshness of level 4 are not recommended to be consumed. In an embodiment, it is also possible to select only the candidate food materials that have lower freshness but are still edible as the target food materials. For example, it is feasible to consider only the food materials with the freshness of level 3 as the target food materials.

The present disclosure provides multiple ways to acquire the freshness of a candidate food material. One way is to obtain a learning model in advance and then use the learning model to acquire the freshness of the candidate food material. In an embodiment as shown in FIG. 2, on the basis of the embodiment as shown in FIG. 1, step S11 may include the following steps:

S21, for each of the candidate food materials, inputting a feature of the acquired picture of each of the candidate food material into the learning model corresponding to the biological category of the candidate food material, and comparing the feature of the acquired picture of each of the candidate food material with a feature of the pre-stored food material pictures in the learning model, to obtain a first freshness level of the candidate food material; and

S22, determining the freshness of the candidate food material based on the first freshness level.

The learning model is obtained by learning from a plurality of sample pictures of the candidate food material that are labeled with the first freshness level. The first freshness level is inversely proportional to the degree of freshness of the food material. The higher the first freshness level, the staler the food materials are. The lower the first freshness level, the fresher the food materials are.

To train and obtain the learning model corresponding to each of the biological categories, various vegetables available on the market can be sampled. For the same vegetable, pictures of different first freshness levels are taken respectively as samples. Taking celery as an example, a large number of pictures of celeries having freshness of level 1, level 2, level 3 and level 4 are collected as training samples. By taking these training samples as input and the first freshness levels corresponding to the samples as output, deep learning training is performed, in order to obtain the corresponding learning model. For various vegetables of the same biological category, the same model is used for deep learning training to obtain the learning model of the corresponding the biological category.

For each candidate food material, after acquiring its picture and determining its biological category, the corresponding learning model may be selected according to the biological category to which the candidate food material belongs. Then the features of the acquired picture of the candidate food material is input into the selected corresponding learning model to acquire the first freshness level of the candidate food material.

The recipe recommendation method according to this embodiment acquires the first freshness level of the candidate food material by collecting a picture of each candidate food material and inputting the feature of the collected picture of the candidate food material into the learning model corresponding to the biological category of the candidate food material, and thus ensures the accuracy of the recognition of the freshness of the candidate food material.

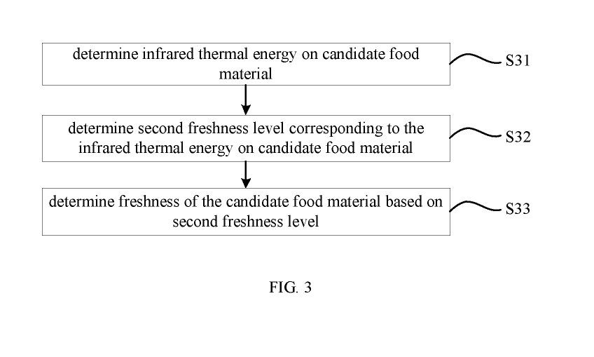

With the increasing of the preservation time of the vegetable, some microorganisms may grow on the surface of the vegetable. The activity of the microorganisms on the vegetable surface will produce heat. The less fresh the vegetable, the more microorganisms on the vegetable surface will grow, and the more the produced heat is. Therefore, there is a positive relationship between the freshness level of the vegetable and the amount of heat generated on the vegetable. A positive relationship means that the freshness level of the candidate food material monotonically increases or decreases as the infrared thermal energy increases or decreases. Thus, as another possible implementation to acquire the freshness of the candidate food material, the freshness of the vegetable can be determined based on the thermal energy produced by the microorganisms on the vegetable. As shown in FIG. 3, on the basis of the embodiment shown in FIG. 1, step S11 may include the following steps:

S31, determining the infrared thermal energy emitted by each of the candidate food material;

S32, determining a second freshness level corresponding to the infrared thermal energy of each of the candidate food material based on a positive relationship between the infrared thermal energy and the second freshness level of the candidate food material; and

S33, determining the freshness of the candidate food material based on the second freshness level.

As mentioned above, the less fresh the vegetable, the more the produced thermal energy will be. Therefore, in this embodiment, the infrared thermal energy emitted by each of the candidate food materials may be determined firstly, and then the second freshness level of the candidate food materials may be determined based on the infrared thermal energy.

As an example, infrared sensors may be installed in the refrigerator such that the infrared thermal energy emitted by each of the candidate food materials may be acquired using the infrared sensors.

The more the infrared thermal energy emitted by the food material, the less fresh the food material is, and the higher the second freshness level of the food material is. Therefore, in this embodiment, the positive relationship between the infrared thermal energy and the second freshness level may be set and stored in advance. The positive relationship between the infrared thermal energy and the second freshness level is shown by the formula 1 below.

$$\{ \begin{matrix}M \leq th 1 , & the second freshness level is level 1 ; \\ th 1 < M \leq th 2 , & the second freshness level is level 2 ; \\ th 2 < M \leq th 3 , & the second freshness level is level 3 ; \\ M > th 1 , & the second freshness level is level 4\end{matrix} . \qquad ( 1 )$$

where M represents the infrared thermal energy value emitted by the microorganisms on the food material, and th1, th2, th3 and th4 are the preset infrared thermal energy thresholds, with th1<th2<th3<th4. It should be understood that th1, th2, th3 and th4 may be any reasonable, artificially set infrared thermal energy thresholds.

In this embodiment, after acquiring the infrared thermal energy of each candidate food material, the second freshness level of each candidate food material can be determined according to the positive relationship between the infrared thermal energy and the second freshness level as shown in formula (1), thereby the freshness of each candidate food material is determined.

By determining the infrared thermal energy of each candidate food material, the method can determine the freshness corresponding to the infrared thermal energy of each candidate food material according to the positive relationship between infrared thermal energy and freshness level, and can ensure the accuracy of the acquired freshness of the food material.

It should be understood that the above two ways to acquire the freshness of a candidate food material can be applied individually. However, in order to avoid the deviation caused by using a single way to acquire the freshness of the food material and to further improve the accuracy of the acquired freshness of the food material, in a possible implementation of the embodiment of the present disclosure, the above two ways to acquire the freshness of the food material may also be combined, where the freshness of the food material is ultimately determined based on the combined result of the above two ways.

In an exemplary embodiment, it is assumed that the score of the freshness of level 1 is set as 1, the score of the freshness of level 2 is set as 2, the score of the freshness of level 3 is set as 3, and the score of the freshness of level 4 is set as 4. The score of the first freshness level obtained by the way using the camera is set as cscore which owns a weight of q1. The score of the second freshness level obtained by the way using the infrared sensor is rscore which own a weight of q2. In an embodiment, q1=q2=0.5. In this way, the score of the freshness of the food material is shown as formula (2).

score=q₁*cscore+q₂*rscore (2)

The obtained result is rounded off to give the final freshness score. The freshness score reflects the freshness of the food material. Therefore, a score of the recipe corresponding to the food material can be determined from this score, so that the recipe can be recommended accordingly. For example, in an example, the method includes indicating a food material with a score of 4 (i.e., a food material determined to be inedible) and preferentially recommending to the user a recipe of a food material with a score of 3 (i.e., a food material less fresh but still edible).

The deviation of the freshness obtained by a single way can be reduced by ultimately determining the freshness of the food material by combining the two ways, and therefore the accuracy of the acquired freshness of the food materials is further improved.

In this embodiment, after acquiring the freshness of one or more candidate food materials, the candidate food materials may be classified as inedible food materials that have low freshness resulting inedibility and acceptable target food materials according to the acquired freshness of the candidate food materials (step S12). In an embodiment, for example, when the freshness of the candidate food materials is set into 4 freshness levels, the candidate food materials having freshness levels of 1 to 3 are selected as the target food materials because it is not recommended to consume food materials having the freshness of level 4. In another embodiment, it is also possible to select the food material with a low freshness but still edible as the target food material. That is, the candidate food material with the freshness of level 3 is selected as the target food material. After the target food materials are selected, candidate recipes corresponding to the target food materials are further retrieved from a database with regard to each of the target food materials, to generate a set of candidate recipes (step S13). In this embodiment, according to the acquired candidate recipes of the plurality of food materials, the recipes of each target food material may be combined to generate the set of candidate recipes.

Then, the score of the candidate recipe can be calculated, which is used to indicate the degree to which the candidate recipe is recommended (step S14). In this embodiment, each candidate recipe in the set of candidate recipes may be scored by taking into account various factors. For example, the candidate recipe may be scored according to the user's preference on dishes with the highest score given to the candidate recipe that matches the user's preference on dishes to the highest degree. The factors taken into account also include the degree of matching of the user's preferences on tastes, the degree of coincidence of the nutrition with the dishes consumed in a certain period of time, the number of appearances of the candidate recipe in the set of candidate recipes, and the like. The use of these factors is described in more detail below.

After the score of the candidate recipe is calculated, determine a recommended recipe based on the score of the candidate recipe in the set of candidate recipes (step S15). For example, the candidate recipe with the highest score may be determined as the recommended recipe based on the score of the candidate recipe. After determining the recommended recipe, recommend the recommended recipe to the user (step S16). As an example, a display panel may be provided on the door of a refrigerator, and the recommended recipe may be displayed to the user through the display panel.

The recipe recommendation method of the present disclosure is capable of generating recipes according to the freshness of the user's food materials at hands and recommending them to the user, while avoiding the waste of the food materials. This method recommends recipes of food materials with lower freshness but still edible to users, making the user preferentially consume these food materials so as to prevent these food materials from being preserved too long and decomposed therefore becoming inedible, thus solving the technical problem of wasting food materials.

In addition to determining the recommended recipe based on the freshness of the food materials, the present disclosure also provides a method of recommending recipes based on other factors.

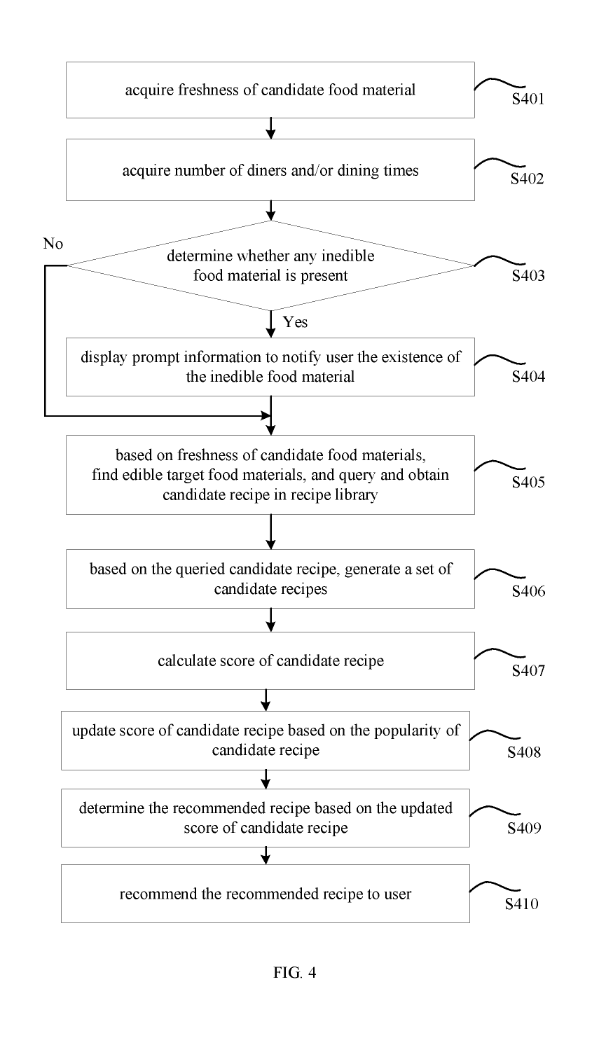

FIG. 4 is a schematic flow chart of a recipe recommendation method according to an embodiment of the present disclosure. This embodiment takes into account factors such as the number of diners, the dining time, the popularity of the recipe, and the like. It should be understood that the execution order of the steps recited herein is merely exemplary. These steps can be performed in other suitable orders.

As shown in FIG. 4, the recipe recommendation method may include the following steps:

S401, acquiring the freshness of a plurality of candidate food materials;

S402, acquiring at least one of a number of diners and a dining time entered by a user;

S403, determining whether any inedible food material is present;

S404, displaying prompt information to notify the user the existence of the inedible food material;

S405, querying and acquiring at least one target food material among a plurality of candidate food materials according to the freshness of the plurality of candidate food materials, and querying and acquiring a candidate recipe for each of the target food materials in a recipe library corresponding to at least one of the number of diners and the dining time;

S406, generating a set of candidate recipes according to the candidate recipes of the target food materials;

S407, calculating score of the candidate recipe;

S408, for each candidate recipe, updating the score of the candidate recipe based on the popularity of the candidate recipes;

S409, determining a recommended recipe according to the updated score of the candidate recipe; and

S410, recommending the recommended recipe.

The above recipe recommendation method will be described in detail below.

In an embodiment of the present disclosure, the freshness of the plurality of candidate food materials may specifically be acquired by using the method described in the above embodiments (step S401). For the sake of conciseness, this will not be described in detail here.

In order to meet the dining requirements of the user, at least one of the number of diners and the dining time of the users can be further acquired (step S402). For example, an input panel may be provided on the refrigerator, and at least one of the number of diners and the dining time may be input by the user through the input panel. Alternatively, a control terminal may be provided for the refrigerator to input at least one of the number of diners and the dining time through an input interface of the control terminal.

By taking into account the number of diners and the dining requirements of the user, it is able to ensure the match between the recommended recipe and the actual requirements of the user, therefore further improving the appropriateness of the recipe recommendation.

It should be noted that the order of execution of step S401 and step S402 are not constant. They could be performed concurrently or in any sequence. In the example of the present embodiment, step S402 is described by way of example as performed after step S401. This example cannot be construed as a limitation of the present disclosure.

Whether any inedible candidate food material is present may be determined according to the aforementioned method for acquiring the freshness of the candidate food material (S403).

Thresholds dividing the freshness levels of the food materials can be set in advance. For example, the threshold can be set to the freshness of the candidate food materials that should recommended to be consumed immediately. For example, when the freshness of the food materials is divided into four levels, the food materials with the freshness of level 1 or 2 are set as relatively fresh and can be stored in continuation. The food materials with the freshness of level 3 are set as less fresh and should be recommended to be consumed immediately. The food materials with freshness of level 4 are set as the least fresh food material and should not be recommended for consumption. In this case, when the freshness of a food material is level 4, this food material can be determined as inedible.

In this embodiment, after acquiring the freshness of the plurality of candidate food materials, it can be determined whether any inedible candidate food material exists. When there is an inedible candidate food material, step S404 is performed; otherwise, step S405 is performed.

If there is any inedible food material, prompt information is displayed to notify the user the existence of the inedible food material (step 404).

In this embodiment, when there is an inedible candidate food material, the prompt information may be displayed on a display panel on the door of the refrigerator to notify the user of the candidate food material having a freshness level higher than the threshold. As an example, a picture of the candidate food material having a freshness level higher than the threshold may be displayed on the display panel. Meanwhile, text information such as “The food material is no longer edible, please discard!” is displayed to notify the user with the inedible candidate food material, protecting the user from physical discomfort due to eating stale food materials by accident, which is advantageous to the user's health. At the same time, it is also avoided that the stale food materials contaminate the fresh food materials.

Based on the freshness of the plurality of candidate food materials, the edible target food materials are found among the candidate food materials. In the recipe library corresponding to at least one of the number of diners and the dining time, a candidate recipe for the target food material is queried and obtained (step S405). In an embodiment, only the food materials with relatively low freshness are queried among the target food materials at step S405. In another embodiment, all edible food materials are queried at step S405. For example, the stale target food materials can be queried according to the freshness of the candidate food materials, that is, the candidate food material with a higher freshness level are selected, and then the candidate recipes of these food materials are queried and obtained from the recipe library corresponding to at least one of the number of diners and the dining time. Then, based on the queried candidate recipes, generate a set of candidate recipes (step S406).

In this embodiment, the candidate recipes of the plurality of target food materials may be summed to generate the set of candidate recipes. After the candidate recipes are obtained, calculate the score for each of the candidate recipes based on the freshness of the food materials (step S407).

In order to recommend the recipe according to the user's preference on the dishes, in this embodiment, the scores of the candidate recipes are updated for each of the candidate recipes based on the popularity of the candidate recipes (step S408).

Step S408 may be designed such that the refrigerator uploads the recipes selected by all the users from the recommended recipes to the server, and for the same recipe, the server counts the number of times the recipe is selected by all the users.

Therefore, in this embodiment, for each candidate recipe, the number of times the recipe is selected by all the users can be acquired from the server, and in turn the popularity of the recipe can be determined according to the number of times that the recipe is selected by all the users. If a recipe is selected for a larger number of times, the recipe is determined to have a higher popularity. Further, depending on the popularity of the candidate recipe, the score of the candidate recipe may be determined. In particular, a recipe with a high degree of popularity may be given a high score and a recipe with a low degree of popularity may be given a low score. The score of the candidate recipe is then updated (step S408) for subsequently recommending the recipes based on the updated score.

In order to further improve the appropriateness of recipe recommendation, the score of the candidate recipe can be updated in terms of different aspects. In particular, the score may be updated based on a matching degree between the candidate recipe and the user's preference on tastes.

When the score is updated according to the matching degree between the candidate recipe and the user's preference on tastes, weights for a plurality of tastes in the taste dimension are acquired firstly. The weights are determined by learning from historical recipes in terms of multiple tastes. Then, a first correction value is obtained by a weighted calculation performed according to the weights of the plurality of tastes in the taste dimension and a matching degree between the tastes of candidate recipe and the corresponding tastes in the taste dimension. The first correction value is used to indicate the matching degree between the tastes of the candidate recipe and the user's preference on tastes. The corrected score is derived by multiply the score by the first correction value.

The taste dimension can usually be divided into seasoning tastes and cooking technique tastes. Seasoning tastes include acidity, sweetness, bitterness, pungency, saltiness, etc. Cooking technique tastes include frying, sauteing, steaming, boiling, etc. Among many tastes, the user can choose some of them to build the taste dimension.

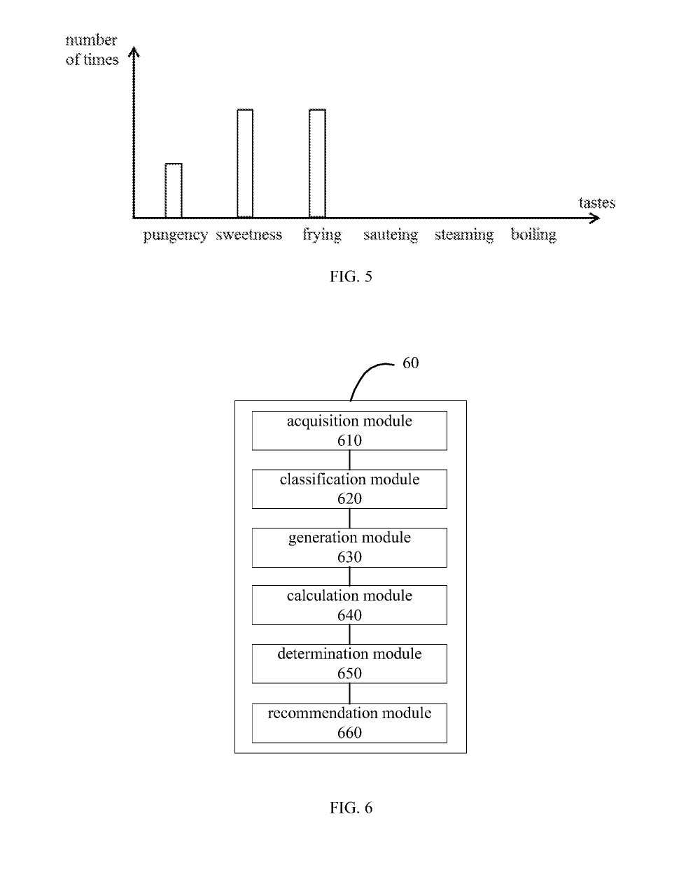

In an embodiment, the user may set the taste dimension to include six tastes comprising pungency, sweetness, frying, sauteing, steaming and boiling. It should be understood that the above embodiment is only an example, and that the taste dimension can also add or delete other various tastes. Taking the above embodiment as an example, historical data may be statistically accumulated according to the above six tastes and a histogram of user historical data may be built. FIG. 5 is an example of the taste dimension. As shown in FIG. 5, the horizontal axis represents the tastes, and the vertical axis represents the statistical result corresponding to different tastes. Assuming that the statistical result of pungency, sweetness, frying, sauteing, steaming and boiling are n1, n2, n3, n4, n5 and n6 respectively, the weights of each taste can be further calculated. Taking pungency as an example, the weight for pungency is shown as formula (3).

$${w}_{1} = \frac{{n}_{1}}{{n}_{1} + {n}_{2} + {n}_{3} + {n}_{4} + {n}_{5} + {n}_{6}} \qquad ( 3 )$$

The method according to this embodiment may be designed such that when recipes are stored, the matching degree between the tastes of each of the recipes and the taste dimension is stored at the same time. The matching degree can be artificially assessed by the designer or the food specialist or even the user per se in terms of the tastes for the same recipe. The higher the matching degree of the recipe with a certain taste, the higher the matching degree is, and the higher the coincidence for that certain taste is. For example, for the taste of pungency, if a dish is not spicy at all, the coincidence with the taste of pungency is zero. If the pungency is lower than the user's requirement on pungency, the coincidence for pungency is 0.5. If the user's requirement on pungency is exactly met, the coincidence is 1. If beyond the user's requirement on pungency, the coincidence can also be set between 0-1, since the value of coincidence can be set by the user per se. In an embodiment, if the user does not like spicy at all, it may set a mild flavor as 1 and the most spicy flavor as 0, with respect to pungency.

Furthermore, in this embodiment, the first correction value may be obtained by a weighted calculation performed according to the weights of the plurality of tastes and the matching degree between the tastes of the candidate recipe and the corresponding tastes in the taste dimension. For example, assuming the tastes of a dish is slightly spicy, with a proper sweetness, and cooked with a sauteing technique, it can be considered that this dish has a pungency coincidence of 0.5, a sweetness coincidence of 1, and a sauteing coincidence of 1, and the coincidences of the remaining tastes are zero. Thus the first correction value w of this dish is:

$$w = {w}_{1} * 0.5 + {w}_{2} * 1 + {w}_{4} * 1 = \frac{{n}_{1} * 0.5 + {n}_{2} * 1 + {n}_{4} * 1}{{n}_{1} + {n}_{2} + {n}_{3} + {n}_{4} + {n}_{5} + {n}_{6}}$$

The updated score is obtained by multiplying the score of the candidate recipe by the first correction value. It should be noted that the above method of calculating the first correction value is merely an exemplary method. The first correction value may be calculated by other suitable methods.

By using the weights of different tastes in the taste dimension to effectively reflect the user's preference on tastes and correcting the score of the candidate recipe based on the taste dimension, the accuracy of the score can be improved and, therefore the appropriateness of the recipe recommendation can be improved as well.

In an embodiment, the score may also be updated according to a nutrition overlap degree between the historical recipe within a certain period of time and the candidate recipe. It specifically includes subtracting the nutrition overlap degree of the candidate recipe from the score of the candidate recipe to obtain the updated score of the candidate recipe.

The period of time can be set by the users according to their personal needs. It may be three days, five days, one week or the like. This disclosure is not limited thereto.

In an embodiment, the refrigerator may record the historical recipes selected by the user and record the specific time at which these historical recipes are selected by the user within the period of time. Then, the candidate recipe is matched with the historical recipe. When the historical recipe is the same as a certain candidate recipe, the nutrition overlap degree of the candidate recipe is determined according to the recorded time when the historical recipe was selected. The closer the selection time to the current date, the higher the nutrition overlap degree of the candidate recipe is. The updated score is obtained by subtracting the nutrition overlap degree of the candidate recipe from the score of the candidate recipe. It is recognized that the score of the candidate recipes that is recently consumed will be greatly reduced.

By taking into account the recipes recently consumed and reducing their score, the recipes that have not been consumed recently can be recommended preferentially to the user to ensure a balanced nutrition is taken by the user and to avoid the repeating of the recipes recently consumed.

In an embodiment, the score may be updated based on the number of times that a candidate recipe appears in the sets of candidate recipes. It specifically includes that determining a second correction value of the candidate recipe according to the number of times that the candidate recipe appears in the sets of the candidate recipes of the plurality of target food material; and summing the score and the second correction value to obtain a corrected score.

How to update the score with the number of times the candidate recipe appears in the set of candidate recipes is illustrated below. In an embodiment, assume that the freshness levels of the edible target food materials are level 1-3. The target food materials are cucumber, cauliflower and tomato, and the recognized freshness level of each of the three candidate food materials is: level 1 for the cucumber, level 2 for the cauliflower, and level 3 for the tomato. The candidate recipes obtained from the recipe library are:

cucumber: A={[1 recipe a1]; [1 recipe a2]; [1 recipe a3]; . . . }

cauliflower: B={[2 recipes b1]; [2 recipes b2]; [2 recipes b3]; . . . }

tomato: C={[3 recipes c1]; [3 recipes c2]; [3 recipes c3]; . . . }

where 1, 2, and 3 represent the scores corresponding to the freshness of cucumber, cauliflower, and tomato, respectively.

In this embodiment, when updating the score according to the number of times that the candidate recipe appears in the set of candidate recipes, if the candidate recipe appears multiple times, the base score, which is the highest score of the candidate recipe among its scores in each set, is updated. With reference to the above example, it is assumed that the recipe b2 and the recipe c3 represent the same recipe (e.g., the sautéed tomato with cauliflower), since the freshness score of the recipe c3 is 3 and the freshness score of the recipe b2 is 2, the freshness score of the recipe c3 is defined as the base score. If the growth score for the candidate recipe repeating in appearance once is δ, the corrected score of the recipe c3 is (3+δ).

By scoring food materials with different freshness, in particular, giving a high score to the food materials having a high freshness level, and defining the highest score as a base score when the recipe appears multiple times and correcting the base score according to the number of appearances of the recipe, the waste of the food materials can be avoided and the accelerated consumption of the existing food materials is facilitated.

After updating the scores of the recipes, determine the recommended recipe based on the updated scores of the candidate recipes in the set of candidate recipes (step S409). In this embodiment, one or more recipes with larger score after updating the score may be determined as the recommended recipes. Then, recommend the recommended recipe to the user (step S410).

In an embodiment, after determining the recommended recipe, the recommended recipe may be recommended to the user. Optionally, in a possible implementation of the embodiment of the present disclosure, after recommending the recommended recipes to the user, the target recipe selected by the user from the recommended recipes may be acquired and added to the historical recipes. Then re-learn from the historical recipes to update the weights of multiple tastes of the taste dimension. By re-learning from the historical recipes selected by the user, the weights of different tastes in the taste dimension can be optimized so that the weight of the taste dimension can accurately represent the current taste of the user, thereby further improving the appropriateness of the recipe recommendation.

The present disclosure also provides a recipe recommendation apparatus.

FIG. 6 is a schematic structural diagram of a recipe recommendation apparatus according to an embodiment of the present disclosure.

As shown in FIG. 6, the recipe recommendation apparatus 60 includes an acquisition module 610, a classification module 620, a generation module 630, a calculation module 640, a determination module 650, and a recommendation module 660.

In an embodiment, the acquisition module 610 is configured to identify the biological category of the candidate food material and acquire the freshness of the candidate food.

Optionally, in a possible implementation according to an embodiment of the present disclosure, the acquisition module 610 is specifically configured to acquire a picture of each candidate food material, and determine the biological category to which each candidate food material belongs. The acquisition module 610 inputs the feature of the picture of each candidate food material into the learning model corresponding to the biological category of the candidate food material to obtain the freshness of the candidate food material. The learning model is obtained by learning from a plurality of sample pictures of the candidate food materials that are labeled with the first freshness levels.

Optionally, in a possible implementation according to an embodiment of the present disclosure, the acquisition module 610 is specifically configured to determine the infrared thermal energy emitted by the microorganisms on each of the candidate food materials, and determine, based on a positive relationship between the infrared thermal energy and the freshness, a second freshness level corresponding to the infrared thermal energy emitted by the microorganisms on each candidate food material to determine the freshness of the candidate food material.

Optionally, in a possible implementation according to an embodiment of the present disclosure, the acquisition module 610 is specifically configured to determine the freshness of the candidate food material by performing a weighted calculation on the first freshness level and the second freshness level of the candidate food material.

In an embodiment, the classification module 620 is configured to classify the candidate food materials into the target food materials and the inedible food materials according to the freshness of the candidate food materials.

In an embodiment, the generation module 630 is configured to acquire a candidate recipe of the target food materials to generate a set of candidate recipes.

In an embodiment, the calculation module 640 is configured to calculate a score for each of the candidate recipes, the score indicating a degree to which a candidate recipe is recommended.

In an embodiment, the determining module 650 is configured to determine the recommended recipe according to the score of the candidate recipe in the set of candidate recipes.

In an embodiment, the recommendation module 660 is configured to recommend the recommended recipe.

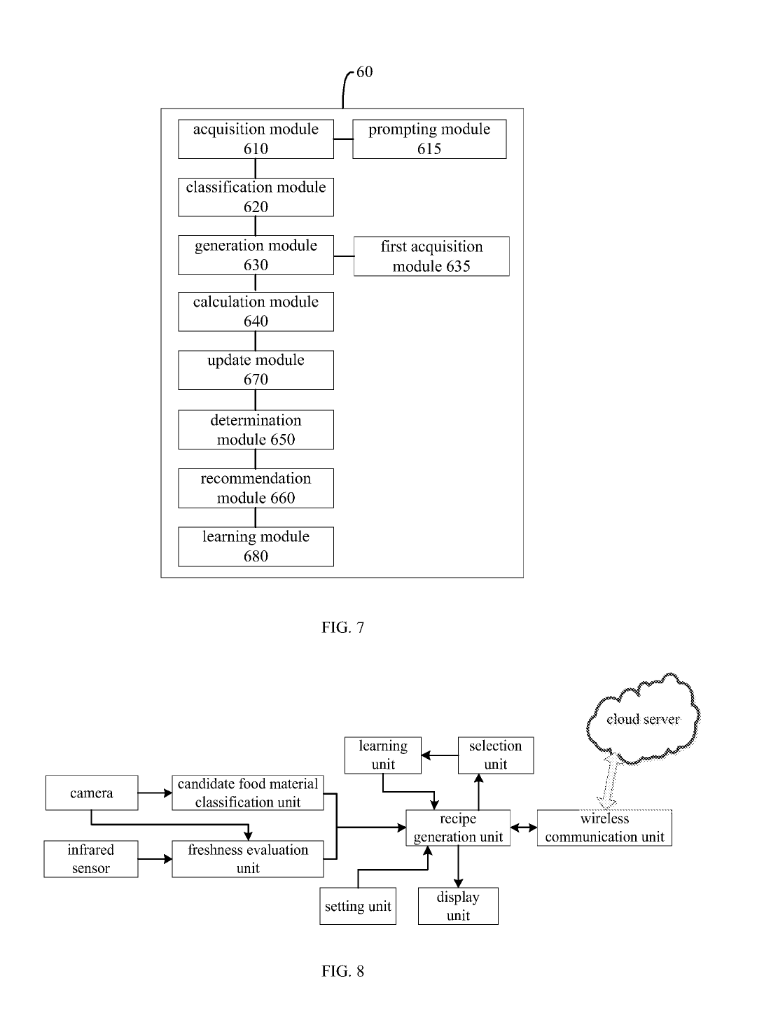

Further, as shown in FIG. 7, in a possible implementation of the embodiment of the present disclosure, the recipe recommendation apparatus 60 may further include an update module 670 on the basis of the embodiment shown in FIG. 6. In an embodiment, the update module 670 is configured to update the score according to the number of times the candidate recipe appears in the set of candidate recipes. In another embodiment, the update module 670 is configured to update the score according to the nutrition overlap degree between the historical recipe within a certain period of time and the candidate recipe. The historical recipe is a recipe selected by the user from the recommended recipes that have been recommended. In yet another embodiment, the update module 670 is configured to update the score according to the matching degree between the candidate recipe and the user's preference on tastes. It should be understood that the update module 670 may update the score based on one or more of the number of times that the candidate recipe appeared in the set of candidate recipes, the nutrition overlap degree between the historical recipe within a certain period of time and the candidate recipe, the matching degree between the taste of the candidate recipe and the user's preference on tastes, as well as other factors.

When the update module 670 is configured to update the score according to the matching degree between the candidate recipe and the user's preference on tastes, the update module 670 specifically performs the following steps: acquiring a weight of a taste in the taste dimension (the weight is determined by learning from the historical recipes in terms of multiple tastes); determining a first correction value of the score of the candidate recipe by performing a weighted calculation performed based on the overlap degree between the taste dimension and the taste of the candidate recipe (the first correction value indicates the matching degree between the candidate recipe and the user's preference on tastes), and correcting the score of the candidate recipe by multiplying the score by the first correction value.

When the update module 670 is configured to update the score according to the nutrition overlap degree between the historical recipe within a period of time and the candidate recipe, the update module 670 specifically performs the following steps: updating the score of the candidate recipe by subtracting the nutrition overlap degree of the candidate recipe from the score of the candidate recipe.

When the update module 670 is configured to update the score according to the number of times the candidate recipe appears in the set of candidate recipes, the update module 670 specifically performs the following steps: determining a second correction score of the candidate recipe according to the number of times that the candidate recipe appears in the set of candidate recipes; and correcting the score of the candidate recipe by summing the score of the candidate recipe with the second correction score.

In an embodiment, the recipe recommendation apparatus 60 may further include a learning module 680. The learning module 680 is configured to acquire a target recipe selected by the user from the recommended recipes; adding the target recipe to the historical recipes; and re-learning from the historical recipes to update the weight of the tastes in taste dimensions.

In an embodiment, the recipe recommendation apparatus 60 may further include a prompting module 615. The prompting module 615 is configured to display prompting information when an inedible food material exists to notify the user to discard the inedible food material in time.

Optionally, in a possible implementation of the embodiment of the present disclosure, as shown in FIG. 7, the recipe recommendation apparatus 60 may further include a first acquisition module 635, configured to acquire at least one of the number of diners and the dining time input by the user. In this case, the generation module 630 is specifically configured to query and obtain the candidate recipe of each target food material in the recipe library corresponding to at least one of the number of diners and the dining time according to the freshness of the plurality of candidate food materials to generate the set of candidate recipes.

It should be noted that the foregoing explanation of embodiments of the recipe recommendation method also applies to the recipe recommendation apparatus of this embodiment, and their principles of implementation are similar and therefore are not repeated here again.

FIG. 8 is a schematic diagram of a recipe recommendation apparatus according to an embodiment of the present disclosure. As shown in FIG. 8, the recipe recommendation apparatus includes a camera, an infrared sensor, a candidate food material classification unit, a freshness evaluation unit, a setting unit, a learning unit, a selection unit, a recipe generation unit, a display unit, a wireless communication unit and a cloud server. The candidate food material classification unit recognizes the pictures collected by the camera and classifies the food materials to obtain the biological category information of the food materials. The freshness evaluation unit evaluates the freshness of the food materials based on the pictures collected by the camera and the data collected by the infrared sensors. The setting unit may be provided with some parameters by the user, such as the dining time and the number of diners, to help the recipe generation unit to recommend the recipe according to the actual dining requirement. The selection unit stores the recommended recipes previously selected by the user. The learning unit determines the preference of the user according to the historical selection of the user. The recipe generation unit recommends the recipes to the user according to the preference of the user and the freshness of the food materials, and displays the recipes on the display unit. The recipe generation unit communicates with the cloud server through the wireless communication unit, and the cloud server stores the user's registration information, the user's preference data and the like, and also provides new recipes for the recipe generation unit. By means of the recipe recommendation apparatus, recipes can be generated according to food materials owned by users at hand, and recommended to the user based on the actual dining requirements and preferences of the user, thus improving the appropriateness of recipe recommendation and avoiding the waste of food materials.

The present disclosure also provides a refrigerator.

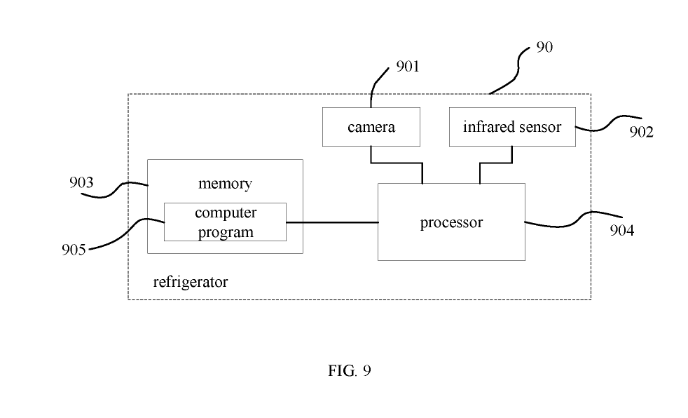

FIG. 9 is a schematic structural diagram of the refrigerator according to an embodiment of the present disclosure.

As shown in FIG. 9, the refrigerator 90 includes a camera 901 and/or an infrared sensor 902, a memory 903, a processor 904, and a computer program 905 stored on the memory 903 and executable on the processor 904. The camera 901 is configured to obtain a picture of each of the candidate food materials. The infrared sensor 902 is used to determine the infrared thermal energy emitted by the microorganisms on each of the candidate food materials. The processor 904 is configured to implement the recipe recommendation method as described in the above embodiments by executing the computer program 905 according to the picture acquired by the camera 901 and/or the infrared thermal energy determined by the infrared sensor 902.

With the recipe recommendation method, the recipe recommendation apparatus and the refrigerator according to the present disclosure, the freshness of a plurality of candidate food materials can be acquired, and the candidate food materials can be classified into target food materials or inedible food materials according to the freshness, and the candidate recipe for each target food material can be obtained to generate a set of candidate recipes. A score of the candidate recipe is calculated, and a recommended recipe is determined based on the score of each candidate recipe, and recommended to the user. Thereby, it is possible to generate a recipe based on the freshness of the currently owned food materials and recommend the recipe to the user, while avoid the waste of the food materials. The present disclosure is able to generate recipes which adopt less fresh, but still edible food materials, therefore recommending the user to preferentially consume these food materials so as to prevent the food materials from being preserved too long and becoming decomposed and inedible, thereby solving the technical problem of the wasting of the food materials.

In order to implement the above embodiments, the present disclosure further provides a non-transitory computer-readable storage medium storing thereon a computer program that, when executed by a processor, implements the recipe recommendation method as described in the aforementioned embodiments.

In order to implement the above embodiments, the present disclosure further provides a computer program product which executes the recipe recommendation method as described in the aforementioned embodiments when the instructions in the computer program product are executed by a processor.

In the description of the disclosure, the description with reference to the terms “an embodiment,” “some embodiments,” “an example,” “a specific example,” or “some examples” and the like means that the specific features, structures, materials, or characteristics described in connection with the embodiment or example are included in at least one embodiment or example of the present disclosure. In the specification, a schematic expression of the above terms is not necessarily directed to the same embodiment or example. Furthermore, the specific features, structures, materials, or characteristics described may be combined in any suitable manner in any one or more of the embodiments or examples. In addition, in case of no contradiction, those skilled in the art may incorporate and combine different embodiments or examples and the features of different embodiments or examples described in the specification.

In addition, the terms “first” and “second” and the like are used for descriptive purposes only and should not be construed as indicating or implying the relative importance or implicitly indicating the number of indicated technical features. Thus, features defined with “first”, “second” and the like may explicitly or implicitly include at least one of the features. In the description of the present disclosure, unless expressly stated otherwise, the definition of “a plurality of” includes the number of at least two, for example, two, three, etc.

Any process or method described in flow charts or otherwise herein may be understood as one or more modules, segments or portions for the code of executable instructions for implementing steps of a customized logic function or process. The scope of the embodiments of the present disclosure includes additional implementations in which functions may be performed in an order not shown or discussed, including a substantially simultaneous or reversed order according to the functions involved, which should be understood by those skilled in the art to which the embodiments of the present disclosure belong.

Logic and/or steps represented in the flow charts or otherwise described herein (which for example, may be a sequenced listing of executable instructions for implementing logic functions) may be embodied in any computer-readable medium for used by or in connection with an instruction execution system, apparatus, or device (such as a computer-based system, a system including a processor, or other system that an instructions may be fetched from an instruction execution system, an apparatus, or a device and executed). So far as this specification is concerned, a “(non-transitory) computer-readable storage medium” may be any apparatus that can contain, store, communicate, propagate, or transport program for use by or in connection with the instruction execution system, apparatus, or device. More specific examples (not a non-exhaustive list) of the computer readable storage medium include electrical connections (electronic devices) having one or more wires, a portable computer disk cartridge (magnetic device), random access memory (RAM), read-only memory (ROM), erasable programmable read-only memory (EPROM or flash memory), optical fiber apparatus, and compact disc read only memory (CDROM). In addition, the computer-readable medium can even be paper or other suitable medium on which the program can be printed, since the program may be obtained in an electronic way and then stored in a computer memory by for example optically scanning the paper or other medium, followed by editing, interpreting or when necessary processing it in other appropriate manners.

It should be understood that portions of the present disclosure may be implemented in hardware, software, firmware, or a combination thereof. In the above embodiments, multiple steps or methods may be implemented in software or firmware stored in memory and executed by a suitable instruction execution system. If implemented in hardware, as in another embodiment, they may be implemented using any one or a combination of the following techniques well known in the art: discrete logic circuits with logic gates for performing logic functions on data signals, application specific integrated circuits with suitable combinational logic gates, programmable gate arrays (PGAs), field programmable gate arrays (FPGAs), and the like.

A person of ordinary skill in the art may understand that all or part of the steps of the methods in the above embodiments may be implemented by a program instructing relevant hardware. The program may be stored in a computer-readable storage medium which, when executed, includes one of the steps of the methods in the embodiments or a combination thereof.

In addition, the functional units in the embodiments of the present disclosure may be integrated in one processing module or exist separately and physically. Two or more units may also be integrated in one module. The aforementioned integrated module can be implemented in the form of hardware or in the form of software functional module. The integrated module may also be stored in a computer readable storage medium when it is implemented in the form of a software functional module and is sold or used as an independent product.

The aforementioned storage medium may be a read-only memory, a magnetic disk, an optical disk, or the like. Although the embodiments of the disclosure have been illustrated and described above, it should be understood that the above embodiments are exemplary and should not be construed as limiting of the disclosure. Those skilled in the art may made changes, modifications, substitutions, and variations to the above embodiments within the scope of the disclosure.

What is claimed is:

1. A recipe recommendation method, comprising steps of:
acquiring a freshness of a candidate food material;
classifying the candidate food material as a target food material or an inedible food material based on the freshness of the candidate food material;
acquiring a candidate recipe corresponding to the target food material to generate a set of candidate recipes;
calculating a score for the candidate recipe, the score indicating a degree to which the candidate recipe is recommended;
determining a recommended recipe based on the score of the candidate recipe in the set of candidate recipes; and
recommending the recommended recipe.

2. The recipe recommendation method of claim 1, further comprising classifying the candidate food material having a lower freshness but still edible as the target food material.

3. The recipe recommendation method of claim 1, further comprising recognizing a biological category of the candidate food material.

4. The recipe recommendation method of claim 3, wherein said recognizing the biological category of the candidate food material comprises:
acquiring a picture of the candidate food material; and
comparing a feature of the acquired picture of the candidate food material with a feature of a pre-stored food material picture to determine the biological category of the candidate food material.

5. The recipe recommendation method of claim 4, wherein said acquiring the freshness of the candidate food material comprises:
inputting the feature of the acquired picture of the candidate food material into a learning model corresponding to the biological category of the candidate food material, and comparing the feature of the acquired picture of the candidate food material with the feature of the pre-stored food material picture in the learning model, to obtain a first freshness level of the candidate food material; and
determining the freshness of the candidate food material based on the first freshness level;
wherein the learning model is obtained by learning from a plurality of sample pictures of the candidate food material that are labeled with the first freshness level.

6. The recipe recommendation method of claim 1, wherein said acquiring the freshness of the candidate food material comprises:
determining an infrared thermal energy on the candidate food material;
determining a second freshness level corresponding to the infrared thermal energy on the candidate food material based on a positive relationship between the infrared thermal energy and the second freshness level of the candidate food material; and
determining the freshness of the candidate food material based on the second freshness level.

7. The recipe recommendation method of claim 4, wherein said acquiring the freshness of the candidate food material comprises:
inputting the feature of the acquired picture of the candidate food material into a learning model corresponding to the biological category of the candidate food material, and comparing the feature of the acquired picture of the candidate food materials with the feature of the pre-stored food material picture in the learning model to obtain a first freshness level of the candidate food material, wherein the learning model is obtained by learning from a plurality of sample pictures of the candidate food material that are labeled with the first freshness level;
determining an infrared thermal energy on the candidate food material;
determining a second freshness level corresponding to the infrared thermal energy on the candidate food material based on a positive relationship between the infrared thermal energy and the second freshness level of the candidate food material; and
determining the freshness of the candidate food material based on the first freshness level and the second freshness level.

8. The recipe recommendation method of claims 1, wherein said calculating the score for the candidate recipe comprises:
determining the score of the candidate recipe corresponding to the candidate food material based on the freshness of the candidate food material.

9. The recipe recommendation method of claim 1, further comprising:
determining a popularity of the candidate recipe; and
updating the score of the candidate recipe based on the popularity.

10. The recipe recommendation method of claim 1, further comprising:
updating the score of the candidate recipe based on a matching degree between the candidate recipe and a user's preference on taste.

11. The recipe recommendation method of claim 10, wherein said updating the score of the candidate recipe based on the matching degree between the candidate recipe and the user's preference on taste comprises:
acquiring a weight of a taste in a taste dimension, wherein the weight is determined by learning from historical recipes in terms of the taste dimension;
determining a first correction value of the score of the candidate recipe by a weighted calculation performed according to an overlap degree of the weight of the taste in the taste dimension and a corresponding taste dimension of the candidate recipe, wherein the first correction value is configured to indicate the matching degree between the candidate recipe and the user's preference on taste; and
correcting the score of the candidate recipe by multiplying the score of the candidate recipe by the first correction value.

12. The recipe recommendation method of claim 11, further comprising:
acquiring a selected recipe selected by the user from the recommended recipe;
adding the selected recipe to the historical recipes; and
re-learning from the historical recipes to update the weight of the taste in the taste dimension.

13. The recipe recommendation method of claim 1, further comprising:
updating the score of the candidate recipe based on a nutrition overlap degree between the historical recipes in a period of time and the candidate recipe, by subtracting the nutrition overlap degree of the candidate recipe from the score of the candidate recipe, wherein the historical recipes are the selected recipes selected by the user from the recommended recipes that have been recommended.

14. The recipe recommendation method of claim 1, further comprising:
updating the score of the candidate recipe based on the number of times that the candidate recipe appeared in the set of candidate recipes by the following steps:
determining a second correction value of the candidate recipe based on the number of times the candidate recipe appeared in the set of candidate recipes of the target food material; and
correcting the score of the candidate recipe by summing the score of the candidate recipe and the second correction value.

15. The recipe recommendation method of claim 1, further comprising:
acquiring at least one of a number of dinners and a dining time entered by a user;
wherein said acquiring the candidate recipe corresponding to the target food material comprises:
querying and obtain the candidate recipe of the target food material in a recipe library corresponding to the at least one of the number of dinners and the dining time.

16. The recipe recommendation method of claim 1, further comprising:
notifying a user of the inedible food material if the inedible food material is present.

17. A recipe recommendation apparatus, comprising:
an acquisition module configured to acquire a freshness of a candidate food material;
a classification module configured to classify the candidate food material as a target food material or an inedible food material based on the freshness of the candidate food material;
a generation module configured to acquire a candidate recipe corresponding to the target food material to generate a set of candidate recipes;
a calculation module configured to calculate a score of the candidate recipe, the score indicating a degree to which the candidate recipe is recommended;
a determination module configured to determine a recommended recipe based on the score of the candidate recipe in the set of candidate recipes; and
a recommendation module configured to recommend the recommended recipe.

18. A refrigerator comprising at least one of a camera and an infrared sensor, a memory, a processor, and a computer program stored on the memory and executable on the processor, wherein
the camera is configured to acquire a picture of a candidate food material;
the infrared sensor is configured to determine an infrared thermal energy on the candidate food material; and
the processor is configured to implement the recipe recommendation method as recited in claim 1 by executing the computer program based on at least one of the picture acquired by the camera and the infrared thermal energy determined by the infrared sensor.

19. A non-transitory computer-readable storage medium storing thereon a computer program which, when executed by a processor, implements the recipe recommendation method as recited in claim 1.

20. A computer program product that executes the recipe recommendation method as recited in claim 1 when an instruction in the computer program product is executed by a processor.
